(19) United States
(12) Patent Application Publication
(10) Pub. No.: US 2010/0021989 A1
(43) Pub. Date: Jan. 28, 2010

(54) COMPOSITIONS AND METHODS OF CONTROLLING AND ADMINISTERING REDOX SPECIFIC FORMS OF DRUGS, FOODS AND DIETARY SUPPLEMENTS

(76) Inventors: Steve Baugh, Broomfield, CO (US); Thomas Hnat, San Diego, CA (US); Alok Nigam, San Diego, CA (US)

Correspondence Address: CATALYST LAW GROUP, APC, 9710 SCRANTON ROAD, SUITE S-170, SAN DIEGO, CA, 92121, US

(21) Appl. No.: 12/510,850

(22) Filed: Jul. 28, 2009

Related U.S. Application Data
(60) Provisional application No. 61/084,178, filed on Jul. 28, 2008.

(51) Int. Cl. C12N 7/06; A01N 43/16; A01N 31/08; A01N 35/00; A01P 1/00

(52) U.S. Cl. 435/238; 514/456; 514/731; 514/734; 514/688

(57) ABSTRACT

The formulations have an antimicrobial, antiviral, and anti-pathogenic composition that combines, in various forms, a redox-active polyphenol, an oxidizing agent, and/or a redox-active, transition metal ion and/or electrochemical potential. The composition relates to methods for decreasing or eliminating the infectivity, morbidity, and rate of mortality associated with a variety of pathogenic organisms and viruses. The present invention also relates to methods and compositions for treating herpes simplex and HIV viruses and drug-resistant bacteria, and for decontaminating areas colonized or otherwise infected by pathogenic organisms and viruses. Moreover, the present invention relates to methods, compositions, electrochemical devices, and storage containers for decreasing the infectivity of pathogenic organisms in pharmaceuticals, medical devices, personal care products, recreational products, and foodstuffs.

### PRIORITY CLAIM

This patent application contains subject matter claiming benefit of the priority date of U.S. Provisional Patent Application Ser. No. 61/084,178 filed on Jul. 28, 2008, accordingly, the entire contents of this provisional patent application is hereby expressly incorporated by reference.

### FIELD OF THE INVENTION

The present invention relates to compositions containing activated forms of polyphenolic compounds, namely the semiquinone and quinone species found in medicines, foods and dietary supplements. The redox state of the composition, and more specifically, target polyphenols within the composition, can be controlled as necessary to deliver the most effective form of polyphenol, depending on the treatment or process. Also related are the methods of processing, identifying, and maintaining the activated species of these compounds.

### BACKGROUND OF THE INVENTION

### Polyphenolic Compounds

(−)-Epigallocatechingallate (EGCG, C₂₂H₁₈O₁₁ Mol. wt.: 458.40; FIG. 1) is the major component of the polyphenolic fraction of green tea (Camellia sinensis). Along with other tea flavonols (catechins), and polyphenols in general, it can bind metals and is an antioxidant that protects cellular components from oxidative damage via free radical scavenging. Many studies have demonstrated the free radical scavenging activities of EGCG, as well as its antiviral (Nakayama et al., 1993; Fassina et al., 2002; Yamaguchi et al., 2002; Kawai et al., 2003; Weber et al., 2003; Chang et al., 2003; Lyu et al., 2005; Song et al., 2005; Hamza et al., 2006), antimicrobial, antimutagenic, antitumorigenic, anti-angiogenic, antiproliferative, and/or pro-apoptotic effects on mammalian cells both in vitro and in vivo (reviewed by Potta et al., 2005). All properties, excluding free radical scavenging activities, are attributed to the generation of reactive oxygen species. Other polyphenols have demonstrated increased protein (Asanuma et al., 2003) and DNA (van Maanen et al., 1988) binding properties in their oxidized states.

Polyphenols can exist in three redox states: fully reduced phenols, partly oxidized radicals (semiquinones), and fully oxidized quinones (FIG. 1). Reduced molecules behave as antioxidants, whereas oxidized polyphenols often behave as pro-oxidants, cycling non-destructively through multiple oxidation states and catalyzing the formation of reactive oxygen species. The oxidized states may also chelate transition metals or catalyze their oxidation. Polyphenols have multiple hydroxyl residues potentially capable of participating in redox reactions (Kilmartin and Hsu, 2003), however not all hydroxyl groups on a molecule may participate in oxidations and reductions under a given set of conditions. These complexities of potential activity are influenced by pH, Eh (redox potential), O₂, the presence of metal ions, proteins and other biological molecules that might react or bind the polyphenols.

Aromatic compounds with phenolic OH groups in adjacent (ortho) positions exhibit enhanced radical stability due to resonance stabilization and intramolecular hydrogen transfer. For these reasons, this feature called a catechol is a site for antioxidant and pro-oxidant activity within a molecule. Additionally, catechols can be redox-cycled through the phenol, semiquinone radical, and quinone forms repeatedly. This is also true for aromatic rings with three hydroxyl groups, called pyrogallols, such as EGCG.

Metal complexation with phenols leads to oxidized species. Phenolic compounds complexed to metals have altered orbitals and singlet/triplet electronic states, and in some cases the complexed phenol is a stabilized radical itself. Radicals are not normally considered stable, but there are multiple ways to have stable radicals or a steady supply of radicals. The most powerful antioxidants are those compounds that are most easily oxidized, which is also an indication of stability of the radical.

Phenolic compounds are antioxidants (reducers) in that they are redox-active molecules in reduced form. They can be subject to oxidation, the loss of an electron, forming radicals and ultimately quinones. Thus, during their own oxidation they reduce biological substrates and protect them. Phenolic molecules behave as antioxidants in the reduced form and often as pro-oxidants in the oxidized radical and quinone forms. They can be reduced in the body, thus causing oxidation of nearby molecules and molecular damage in a pro-oxidant manner. Many polyphenolic compounds can repeatedly cycle non-destructively through the phenol, radical and quinone forms.

In addition to providing a source of stable radicals, quinones are known to complex irreversibly with nucleophilic amino acids in proteins, often leading to inactivation of the protein and loss of function. This is one mechanism thought to provide antiviral and anticancer activity in particular. Many cancer chemotherapy agents act on dividing cells, a process involving radical “sticky ends” that can react with foreign radicals, including medicinal radicals. This activity would lead to damaged DNA and may explain how pro-oxidant chemotherapy treatments act on a dividing cell. In addition, some cancer chemotherapies subject redox-active compounds to light exposure, creating more efficacious oxidized forms in a process called photodynamic therapy.

Using a process of systematic redox-directed drug discovery, modifications of the redox potential of existing medications might possibly demonstrate increased efficacy to both sensitive and resistant strains of pathogens, and lead to patent extensions or new, patentable drug therapies. This technology is appropriate for new drug discovery as well as a re-examination of a large cornucopia of previously approved pharmaceuticals. Adjustment of the redox potential may yield new insights into modes of action for antibiotic and anticancer compounds and other disease treatments.

It is also possible to mine existing data for redox correlations to “rediscover” rejected compounds. Extensive studies have been done with various synthetic drug analogs, yet redox potential hasn't been evaluated as a factor of efficacy. Once the redox potential for each analog is determined, the efficacy data can be re-evaluated for possible correlations to bioactivity profiles of parent compounds based on their redox-sensitive activities. Redox-directed drug discovery of previously rejected compounds could be a rapid and cost-effective strategy for identifying new drug candidates.

The technology is relevant to phenolic compounds in the major representative classes of phenolics. Redox-active compounds falling into these categories include, but are not limited to, phenolics such as cinnamic acids (e.g., caffeic acid, caffeoyltartaric acid, caftaric acid, chlorogenic acid, and rosmaric acid), flavones (e.g., baicalein), flavanones (e.g., exiguaflavanone B and exiguaflavanone D), isoflavones (e.g., genistin, daidzin, glycetin, genistein, daidzein, glycetein, formononetin, and equol), flavan-3-ols (e.g., anthocyanidins, anthocyanins, catechin, epigallocatechin, epigallocatechin gallate, and epicatechin gallate), coumarins (e.g., p-coumaric acid, isocoumarin, and aesculetin), chalcones (e.g., butein), and others such as simple phenolic acids (e.g., ascorbate, p-hydroxybenzoic acid and other benzoic acid derivatives, gallic acid), azo-dyes (e.g., azostilbenes), extensively conjugated phenolics (e.g., anthroaquinones, hypericin and other naphthodianthrones), unsaturated fatty acids (e.g., sorbates), and tocopherols and retinols (e.g., tocopherols, tocotrienols, retinols, and beta-carotene).

Phenolic compounds possessing a C₃ side chain at a lower level of oxidation are classified as essential oils and are often cited as antimicrobial. For example, eugenol is a well-characterized representative found in clove oil. Eugenol is considered bacteriostatic against both fungi and bacteria.

Quinones are oxidized aromatic rings with ketone substitutions. They are ubiquitous in nature and are characteristically highly reactive, oxidizing other molecules to return to their reduced phenolic state. The phenol can then be oxidized again, returning to the quinone state. The redox potential of the particular quinone-hydroquinone pair is very important in many biological systems and may be related to antibiotic efficacy and selectivity.

Flavones are phenolic structures containing one carbonyl group as opposed to the two carbonyls in quinones. The addition of a 3-hydroxyl group yields a flavonol. Flavonoids are also hydroxylated phenolic substances but occur as a C₆-C₃ unit linked to an aromatic ring. They are known to be synthesized by plants in response to microbial infection, and they have antimicrobial activity against a wide array of microorganisms. Their activity may be due to their ability to complex with extracellular and soluble proteins and to complex with bacterial cell walls, similar to the mechanism of quinone antimicrobial activity.

Cinnamic acid and caffeic acids are common representatives of a wide group of phenylpropane-derived compounds which are in the highest oxidation state. The herbs tarragon and thyme both contain caffeic acid, which is effective against viruses, bacteria and fungi. Catechol and pyrogallol both are hydroxylated phenols, shown to be toxic to microorganisms. Catechol has two OH groups, and pyrogallol has three. The sites and number of hydroxyl groups on the phenol groups are thought to be related to their relative toxicity to microorganisms, with evidence that the number and location of phenolic or hydroxyl groups increased toxicity. Thus, phenols that are more highly oxidized would be expected to exhibit a greater toxicity to microorganisms. However, our data and the work of others indicate formulations may become over-oxidized and suffer from diminished efficacy. This reinforces the need for fine redox control and alludes to the differences between radical and quinone forms in some applications.

### Eh and pH Chemistry

Eh-pH-diagrams are commonly used in geochemistry and industrial chemistry to predict ion reactions, stability, and chemical states. The simplest type of these diagrams is based on a chemical system consisting of one element in water solution, such as the sulfur-water system (FIG. 2). The chemical stability area of water is indicated with dotted lines. The upper stability limit of water is based on the redox potential when oxygen generation begins on the anode, and the lower stability limit is based on hydrogen formation on the cathode. The system can contain several types of species, such as dissolved ions and gases, hydroxides, oxides, and polymeric forms (e.g., polysulfides not shown in FIG. 2).

The redox potential (Eh) of the system represents its ability to add (reduction) or remove (oxidation) electrons. The system tends to remove electrons from a species when the potential is high, but this Eh value is specific to a redox pair of ions. High potential conditions may exist near the anode in an electrochemical cell, but it can also be generated with some oxidizing agents. Under reducing conditions when the potential is low, the system is able to supply electrons to the species near a cathode electrode or from chemical reducing agents with a more electronegative potential.

The pH describes the ability of a system to supply (acidify) or remove (basify) protons from the species. For redox-active elements and molecules, Eh and pH are interdependent. Under physiological conditions, the pH of most tissues normally ranges between pH 7-8. However, the stomach maintains an acidic pH, and topical formulations may range from acidic to alkaline to react with external targets. The pH of drug formulations are generally controlled by buffers commonly utilized in the pharmaceutical industry.

The concept of Eh-pH systems has not been developed in the fields of biochemistry and medicinal chemistry. Based on the premises developed in inorganic chemical systems, changing the pH of a dissolved redox-active biological molecule may alter its tendency to act as an antioxidant or pro-oxidant in cellular systems. In addition, the complex aqueous mixtures in cells and tissues may modify reactions predicted in the simple water-single molecule models. Hence, the relationships between redox states, pH, and biological activity of redox-active molecules such as phenolic compounds must be developed empirically.

There is a widely held notion in the fields of medicinal chemistry and pharmacology that redox-active compositions should be delivered to cell and tissue targets in their reduced, antioxidant state. For example, testing EGCG as an anti-HIV-1 agent to inhibit viral glycoprotein gp120 binding to cell CD-4 receptors utilized only reduced EGCG (Hamza et al., 2006). Furthermore, a device does not exist in the art which would enable researchers to formulate either reduced or oxidized compounds, within specific parameters, in order to carry out such experiments. There exists a need in the field to have a device of the present invention in order to measure and develop precise formulations of redox-adjusted compounds for advancements in immune response therapeutics.

Polyphenols and other compounds can be subjected to various redox manipulations involving chemical techniques (Vardosanidze et al., U.S. application Ser. No. 10/500,301). Such chemical manipulations involve the addition of charged molecules, such as amino acids and their derivatives, in an effort to stabilize the redox properties of a given composition. Such a technique, however, relies on chemical manipulation of a composition to alter its redox properties. This results in the distinct disadvantage of having to go through necessary purification steps to rid the composition of the charged molecules needed to perform the chemical manipulation. There exists a need in the field to perform such manipulations in a way such that the composition is left unaffected by the addition of other molecules.

These and other compounds are subject to electrochemical manipulation utilizing a variety of specific techniques including, but not limited to, cyclic voltammetry, linear sweep voltammetry, bulk electrolysis, normal and differential pulse voltammetry, normal/differential pulse polarography, stripping voltammetry, chronopotentiometry and other like techniques as applied by those skilled in the art. One embodiment of this method is described in WO 2007/090096, “Electrochemical methods for redox control to preserve, stabilize, and activate compounds”, by Steve Baugh and Thomas Hnat. This invention, which is used at as a tool in this patent, is herein referred to as a “battery-in-a-bottle.”

Electrochemical techniques can also be used to prepare and stabilize pro-oxidant formulations. In one embodiment, the desired oxidation state of a molecule can be directly controlled, and stabilized. Fine control of oxidation is increasingly important as redox correlation to efficacy increases. It is possible that electrochemistry offers the most accurate and precise control of redox state in solution and in the body. This premise also applies to electrodynamic therapy, a treatment process analogous to photodynamic therapy that relies on electrochemical oxidation rather than light energy.

There exist other chemical, electromagnetic and nanotechnology methods of redox control, alone or in combination, that also allow control of polyphenol redox states. These devices and formulations have not been evaluated or applied to the systematic redox modulation of drugs in discovery, development or treatment phases. As the redox-based theory of medicine continues to emerge and develop, there will be increasing need for all manners of redox control concerning the manufacture, storage and effect of species on localized micro-oxidation environments within the body. These contributions include nanotechnology devices to hold and control the oxidation state, alone or in combination with electromagnetic fields, possibly incorporating time-release properties as well.

There is a present need for the application of methods for stabilizing the redox state during the preparation of a given material so that a desired pH and redox state can be maintained during manufacturing, storage, consumption, administration or use. The present invention describes methods and devices useful for providing foods, beverages, personal care products, cosmetics, nutritional supplements, reagents, analytical standards, medical device formulations, pharmaceutical preparations and drugs with a desired redox state, either reduced or oxidized.

Many products including foods, beverages, personal care products, cosmetics, nutritional supplements, medical device formulations, pharmaceutical preparations and drugs contain polyphenolic structures within their ingredients that can oxidize to destabilize or degrade the compound, formulation or product. The beneficial reduced forms of these molecules could be stabilized electrochemically for extended shelf life. Other products such as chemotherapy dosage forms would benefit from electrochemical stabilization of the oxidized form(s). Many phenolic compounds can be active pro-oxidants (oxidizers) or antioxidants (reducers), depending on manufacturing, storage conditions, and use.

### SUMMARY OF THE INVENTION

The present invention relates to compositions containing the activated forms of polyphenolic compounds, namely the semiquinone and quinone species. Other aspects of the present invention are the methods of processing, identifying and maintaining the activated species of these compounds.

In another aspect, the present invention encompasses polyphenols and antioxidants, which once oxidized to form the semiquinone or quinone could become antibacterial, antifungal and antiviral/anticancer agents. In another aspect, the oxidation states include phenol, semiquinone, quinone, and oligomeric forms (dimers, trimers, tetramers, and larger polymers) of the polyphenolic substances. Additional embodiments include a redox-active formulation, wherein the redox-active semiquinone, quinone, or oligomeric polyphenolic substance is epigallocatechin gallate. Alternatively, the composition is encapsulated in nanoparticles. Alternatively, the redox-active semiquinone polyphenolic substance is epigallocatechin gallate with the IUPAC name, [2,6-dihydroxy-4-[(2R,3R)-5,7-dihydroxy-3-(3,4,5 trihydroxybenzoyl)oxy]chroman-2-yl]phenoxy. Additionally, the composition also comprises an acceptable metal complex and/or metal salt, wherein the metal is copper, iron, magnesium, calcium, or zinc. Yet another aspect of the invention exists, wherein the semiquinone or quinone polyphenolic substance is a cinnamic acid (e.g., caffeic acid, caffeoyltartaric acid, caftaric acid, chlorogenic acid, and rosmaric acid), flavones (e.g., baicalein), flavanones (e.g., exiguaflavanone B and exiguaflavanone D), isoflavones (e.g., genistin, daidzin, glycetin, genistein, daidzein, glycetein, formononetin, and equol), flavan-3-ols (e.g., anthocyanidins, anthocyanins, resveratrol, catechin, epigallocatechin, and epicatechin gallate), coumarins (e.g., p-coumaric acid, isocoumarin, and aesculetin), chalcones (e.g., butein), and others such as simple phenolic acids (e.g., ascorbate, p-hydroxybenzoic acid and other benzoic acid derivatives, gallic acid), azo-dyes (e.g., azostilbenes), extensively conjugated phenolics (e.g., anthroaquinones, hypericin and other naphthodianthrones), unsaturated fatty acids (e.g., sorbates), and tocopherols and retinols (e.g., tocopherols, tocotrienols, retinols, and beta-carotene). Alternatively, the semiquinone, quinone, or oligomeric form of the polyphenolic substance is created by raising the pH from acidic to basic using an acceptable alkaline base. Other embodiments include a method of treating cancer, Alzheimer's disease or a viral, bacterial, or fungal infection in a subject, comprising i) at least one redox-active semiquinone, quinone, or oligomeric form of a polyphenolic substance, and ii) acceptable pharmaceutical excipients. In some embodiments, other bioactive substances may also be present in the formulation. Other embodiments exist, wherein said viral infection is caused by a virus selected from the group consisting of, but not limited to HIV, HPV, HSV-1, HSV-2, MCMV, GPCMV, VZV, EBV, HHV-6, HHV-8, Influenza A, and the Rhinovirus, and further wherein said antiviral activity of said semiquinone, quinone, or oligomeric polyphenolic substance occurs principally by a binding mode of action. Alternatively, the redox-active semiquinone, quinone, or oligomeric polyphenolic substance epigallocatechin gallate is anticancer or antiviral for HIV, HPV, HSV-1, HSV-2, MCMV, GPCMV, VZV, EBV, HHV-6, HHV-8, Influenza A, and the Rhinovirus. Yet another aspect to the invention comprises a nutritional supplement, food, beverage, cosmetic, or surface disinfectant composition comprising: i) at least one redox-active semiquinone, quinone, or oligomeric form of a polyphenolic substance, and ii) acceptable nutritional or cosmetic

### DEFINITIONS

As used herein, the term “disease” refers to a deviation from the condition regarded as normal or average for members of a species, and which is detrimental to an affected individual under conditions that are not inimical to the majority of individuals of that species (e.g., diarrhea, nausea, fever, pain, and inflammation etc). A disease may be caused or result from contact by microorganisms or somatic or genetic conditions, e.g., auto-immune disease or cancer.

As used herein, the term “microorganism” refers to microscopic organisms and taxonomically related macroscopic organisms within the categories of algae, bacteria, fungi (including lichens), protozoa, viruses, and sub-viral agents. The term microorganism encompasses both those organisms that are in and of themselves pathogenic to another organism (e.g., animals, including humans, and plants) and those organisms that produce agents that are pathogenic to another organism, while the organism itself is not directly pathogenic or infective to the other organism. As used herein, the term “pathogen,” and grammatical equivalents, refers to an organism, including microorganisms, that causes disease in another organism (e.g., animals and plants) by directly infecting the other organism, or by producing agents that causes disease in another organism (e.g., bacteria that produce pathogenic toxins and the like).

In some embodiments, the present invention provides compositions and methods suitable for treating animals, including humans, exposed to pathogens or the threat of pathogens. In some embodiments, the animal is contacted with effective amounts of the compositions prior to exposure to pathogenic organisms. In other embodiments, the animal is contacted with effective amounts of the compositions after exposure to pathogenic organisms. Thus, the present invention contemplates both the prevention and treatment of microbiological infections.

As used herein, the term “sample” is used in its broadest sense. In one sense it can refer to animal cells or tissues. In another sense, it is meant to include a specimen or culture obtained from any source, such as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

As used herein, the term “phenol” refers to an aromatic ring of six carbon atoms with at least one —OH group attached. As used herein, the term “polyphenol” which is short for polyhydroxy phenol, refers to a compound with the characteristic six-carbon aromatic ring which has more than one (“poly”) of the —OH groups (also known as hydroxyl groups) attached to it, particularly in ortho- and para-positions to one another. As used herein, the term “polyphenolic” means a group of compounds which includes the anthocyanidins, anthocyanins, aurones, chalcones, flavanols, flavanones, flavones, flavonols, isoflavones or proanthocyanidins. As used herein, the term “EGCG” refers to epigallocatechingallate in any of its oxidation states.

In other embodiments, the present invention provides compositions and methods suitable for decontaminating solutions and surfaces, including organic and inorganic samples that are exposed to pathogens or suspected of containing pathogens. In still other embodiments of the present invention, the compositions are used as additives to prevent the growth of harmful or undesired microorganisms in biological and environmental samples.

As used herein, the term “solution” refers to an aqueous or non-aqueous mixture. As used herein, the term “composition” refers to any oil-in-water emulsion, water-in-oil emulsion, suspension, gel, lotion, ointment, powdered formulation, aqueous or non-aqueous solution or other suitable delivery vehicle.

As used herein, the terms “pharmaceutically acceptable” or “pharmacologically acceptable,” refer to compositions that do not substantially produce adverse allergic or immunological reactions when administered to a host (e.g., an animal or a human). Moreover, in certain embodiments, the compositions of the present invention may be formulated for horticultural or agricultural use. Such formulations include dips, sprays, seed dressings, stem injections, sprays, and mists. As used herein, “pharmaceutically acceptable carrier” includes any and all solvents, dispersion media, coatings, wetting agents (e.g., sodium lauryl sulfate), isotonic and absorption delaying agents, distringants (e.g., potato starch or sodium starch glycolate), and the like.

As used herein, the term “redox state” or “redox potential” means the measure of the tendency of a solution to give up or take up electrons (i.e., to be oxidized or reduced, respectively). The redox potential may also be described as the electron pressure that the electrochemical cell exerts. The redox potential when all components are in their standard states is called the standard redox potential. The redox potential (Eh) is measured electrochemically and expressed in units of electrical potential difference (i.e., volts).

As used herein, the term “redox-active” means any compound or molecule in the reduced form that can be oxidized to form the radical or pro-oxidant form.

As used herein, the term “antioxidant” refers to any compound that is subject to oxidation (loss of an electron) by reacting with radicals, through metal chelation, and by scavenging singlet oxygen among others. As used herein, the term “oxidizing agent” refers to any compound that has the ability, through chemical reaction with another, to cause the loss of electrons. This can also include applied energy such as light, electrical potential and/or electromagnetic fields that promote oxidation.

As used herein, the term “redox cycling” refers to the repeated oxidation/reduction of a compound during radical reactions. The ability of a redox-active compound to be oxidized, reduced again, and oxidized again, etc., allows the compound to create more radicals than one per compound and be “recycled” at the site. Many redox-active compounds are destroyed by oxidation and cannot be recovered or used as a pro-oxidant reactive intermediate.

As used herein, the term “battery-in-a-bottle” refers to an electrochemical device to alter the redox potential of a solution (see International Publication No. WO 2007/090096).

It is well understood by those experienced in the art that antioxidants function as radical scavengers to prevent a composition's deterioration by oxygen and/or other reactive compounds. Antioxidants are presently incorporated into compositions in minute quantities (usually below 0.01% by weight) to prevent the oxidation of plant and animal oils, thereby extending a product's shelf life. It is also currently understood that antioxidants by themselves provide no antibacterial, antifungal or antiviral properties. Antioxidants are always used in conjunction with chemical preservatives or other processes, i.e., heat or radiation, to prevent microbiological degradation. Antioxidants and antimicrobial chemical preservatives act independently of each other. Antioxidants do not function indefinitely, but rather are used up and destroyed in the process to form the pro-oxidant form. Antioxidants can also participate in a repeated oxidation/reduction of a compound during radical reactions, a process called redox cycling.

The use levels for phenols, polyphenols and antioxidants in pharmaceutical, medical device, personal care, foodstuff and other products is typically less than 0.2 weight %. For many compounds, at concentrations below 0.2 weight %, there is minimal or no recognizable antibacterial, antifungal and antiviral activity. However, undissociated (reduced) forms of some compounds are only known to be effective in an acidic medium. They are known to be ineffective in a neutral and basic medium. Examples of these include, but are not limited to, benzoic acid and sodium benzoate, sorbic acid, potassium sorbate, salicylic acid and methyl salicylate, and possibly acetyl salicylate. The oxidized forms of benzoic acid and sodium benzoate are antibacterial at a pH 7 and greater, as well as in acidic solutions. Thus the oxidized forms have a broader antimicrobial pH range.

Some existing products contain redox-active phenols, polyphenols, or antioxidants or antioxidant systems greater than 0.2% by weight, up to 10 to 20 weight %. In all of these products, the function of these active components relates to the absorption of potentially damaging radicals or minimizing the affects of oxygen degradation. None of these products makes claim to the antibacterial, antifungal or antiviral properties of the pro-oxidant redox-active phenols, polyphenols, or oxidized antioxidants present in the formulation.

The nature of auto-oxidation reactions and redox potentials of catechin flavonoids, including EGCG, is partly known. Bors et al. (2000) used electron paramagnetic resonance (EPR) spectroscopy to investigate radical structures obtained after auto-oxidation of the parent compounds in moderately alkaline solutions. EGCG and other catechins yielded predominantly semiquinone structures derived from the parent catechol.

Mochizuki et al. (2002) studied kinetic and mechanistic aspects of auto-oxidation of catechins and found they quantitatively reduced O₂ to H₂O₂. The semiquinone radical was more susceptible to oxidation with O₂ than fully reduced catechins, and the auto-oxidation rate increased with pH. They found the pyrogallol moiety on the B ring was more susceptible to auto-oxidation than the catechol moiety. Cupric ion enhanced auto-oxidation, probably acting as a catalyst. Zhou et al. (2003) also found that cuprous ions promoted oxidation but neither Fe²⁺ nor Fe³⁺ did. Mochizuki et al. surmised that under physiological conditions (at pH 7.4), the auto-oxidation of catechins would be very slow, as judged by the rather low concentrations of dissolved O₂ in tissues (67 μM). In contrast, Su et al. (2003) found that EGCG was completely degraded in 6 h of incubation in open air at pH 7.4 in phosphate buffer at room temperature. At alkaline pH, it degraded in a few minutes. At pH 7.2, reduced EGCG took hours to oxidize to the quinone form and subsequently form dimers, which also were unstable during a 24-h experiment (Sang et al., 2007).

Kilmartin and Hsu (2003) characterized tea polyphenols using cyclic voltammetry and found the various catechins were readily oxidized in the potential range from 50 to 300 mV. EGCG, which has both pyrogallol and a gallate groups, produced peaks in two potential ranges (105 and 213 mV), unlike epigallocatechin that lacks the fourth phenolic (gallate) ring. The potential near 100 mV corresponds to the redox potential of the hydroxyl groups on the B ring.

### BRIEF DESCRIPTION OF THE DRAWINGS

FIG. 1 depicts varying structures and nomenclature of reduced and oxidized EGCG forms. (A) Reduced phenol embodiment; (B) Semiquinone embodiment; (C) and (D) Oxidized quinones.

FIG. 2 shows an example of an Eh-pH diagram based on sulfur speciation in water.

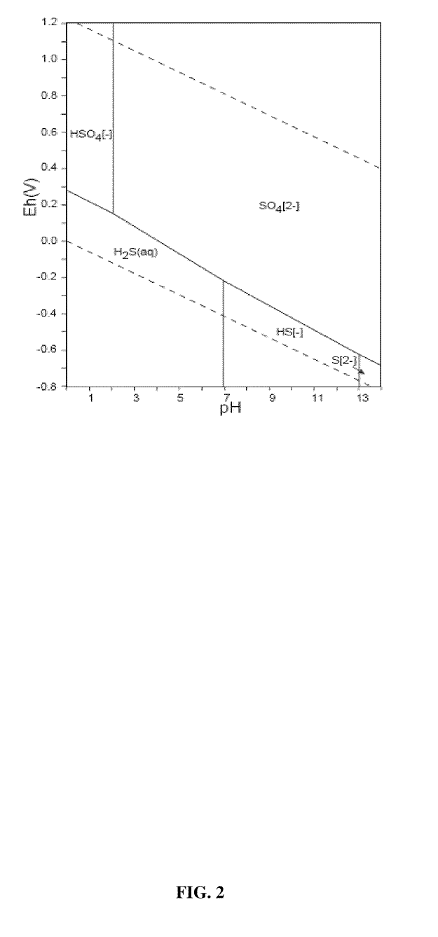

FIG. 3 displays UV-visible absorption spectra of EGCG at different pH's. Examples include (A) fresh solutions of EGCG compared with (B) aged solutions.

FIG. 4 depicts EPR spectrum of EGCG at pH 8.5 in the presence of 0.2 M Mg²⁺, which demonstrates the presence of the semiquinone radical.

FIG. 5 shows the appearance and decay of EGCG radical after basification of the solution over a specific timepoint. (A) is a display with NaOH added and (B) shows the decay of the radical over 30 minutes.

FIG. 6 represents activity of commercial pharmaceuticals versus ECGC against HSV-1 and HSV-2. Non-activated EGCG=reduced; activated EGCG=oxidized. A mixture of the compound and virus was incubated at 37° C. for 1 hour, and then titrated with cells. Concentrations: Benzalkonium chloride (0.00046%), Abreva (100 μM), Acyclovir (10%), EGCG (50 or 100 μM).

FIG. 7 displays a comparison between known antiviral compounds and EGCG (both oxidized and reduced forms) against HSV-1 and -2. Non-activated EGCG=reduced. Activated EGCG=oxidized. EDTA was added with the non-activated EGCG in order to prevent oxidation. Activated EGCG was at pH 7.4 (physiological) and the non-activated EGCG was at pH 6.0.

FIG. 8 shows the effect of various oxidation states of EGCG on (A) HSV-1 and (B) HSV-2. Solution #2 was partially oxidized. Solutions #3 and #4 were oxidized and contained Cu²⁺ and Zn²⁺, respectively.

FIG. 9 depicts the effect of pH on EGCG activity against HSV-1 and HSV-2.

FIG. 10 shows activity of ECGC against HIV-1. Formulation 8\1 was the most oxidized of the four formulations. All four formulations were toxic to cells at higher concentrations.

FIG. 11 depicts NMR spectra of reduced and oxidized EGCG used in Bcl-xL binding assays.

FIG. 12 exhibits Activity of ECGC against multidrug-resistant Staphylococcus aureus.

FIG. 13 shows the effect of electrochemical application on EGCG oxidation at pH 6.0.

FIG. 14 depicts the effects of time and pH modification of EGCG solutions electrochemically treated at pH 6.26, after which the pH was raised to 8.43. The spectra were measured immediately and after 1 day and 7 days.

### DETAILED DESCRIPTION OF THE INVENTION

This invention embodies the pH and redox control of phenolic compounds, with the specific example of EGCG, to determine the most efficacious pharmacological activity, namely antiviral and antibacterial properties at different pH's and Eh's. Examples to support this invention include: 1) UV-visible absorption spectra; 2) electron spin resonance spectra; 3) electrochemical oxidation reactions; 4) in vitro antiviral assays against herpes simplex 1 and 2; 5) in vitro antiviral assays against human immunodeficiency virus HIV-1; 6) in vitro antibacterial assays against multidrug-resistant Staphylococcus aureus; and in vitro anticancer binding assays against Bcl-xL protein.

The present invention covers redox-active phenols, polyphenols, or antioxidants, individually or in combination, at use levels between 0.2% and 30% by weight and preferably between 0.5% and 12%, or combined with other redox-active phenols, polyphenols or antioxidants to form pro-oxidant systems that exhibit antibacterial, antifungal, antiviral, or anticancer activity. Combined redox-active phenols, polyphenols or antioxidants at use levels between 0.01% and 40.0% by weight, preferably between 0.5% and 15.0% by weight, are contemplated to exhibit antibacterial, antifungal, antiviral, or anticancer properties but will vary depending on the composition and its application.

Concentration of the oxidizing agent should be between 0.001% and 10.0% by weight, preferably between 0.05% and 2.0% by weight. Concentration of the redox-active transition metal ion (catalyst) should be between 0.01% and 5.0% by weight, and preferably between 0.05% and 1.0% by weight are contemplated.

### Example 1

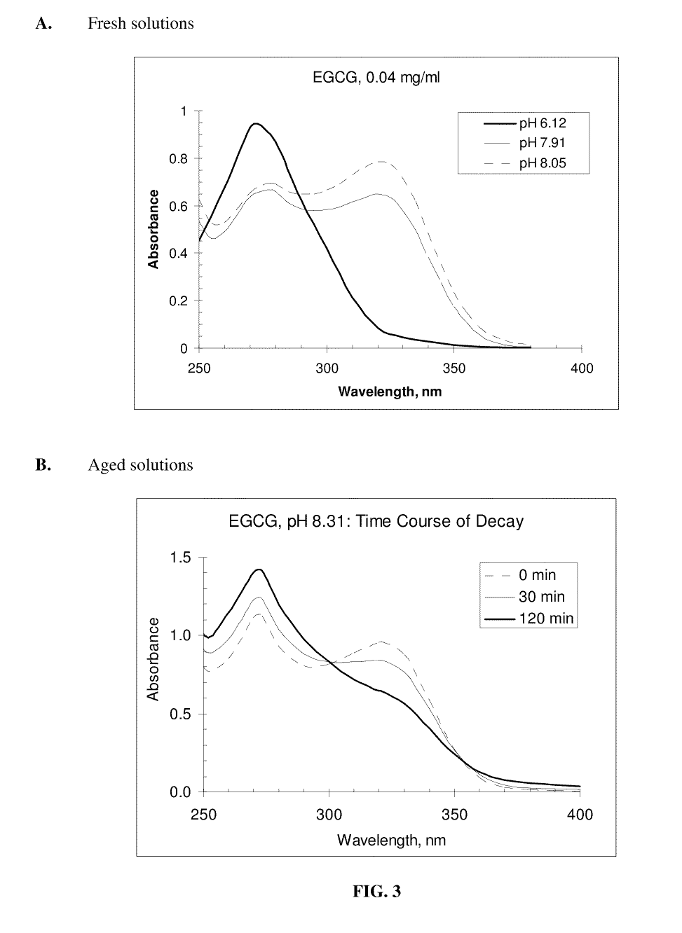

UV-Visible Absorption Spectra vs. pH: EGCG solutions undergo an immediate spectral absorption shift as the pH increases from acidic to alkaline, with a change noted at pH 7 when a peak at 320 nm appears and the dominant phenol peak at 272 nm begins to decrease (FIG. 3A). The 320 nm peak likely represents the absorbance of the quinone form of EGCG. When produced by simply raising the pH of the solution, the 320 nm oxidation peak is labile under air and disappears within hours (FIG. 3B). However, if the alkaline solution is re-acidified within minutes, the reaction is reversible. It is spontaneous with pH change and does not require a catalyst.

### Example 2

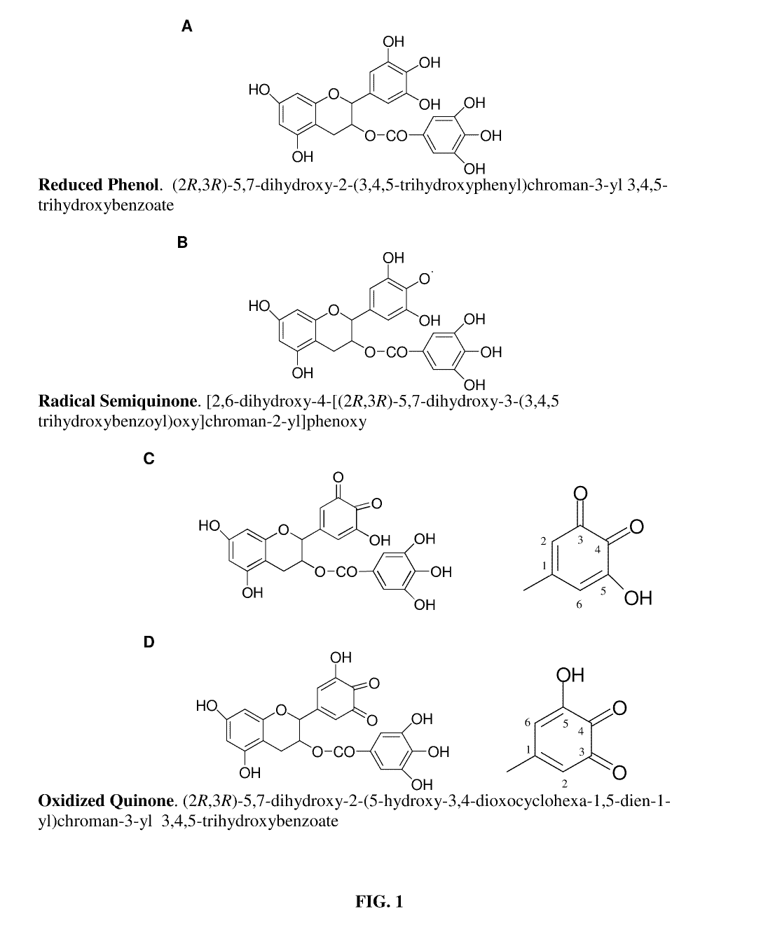

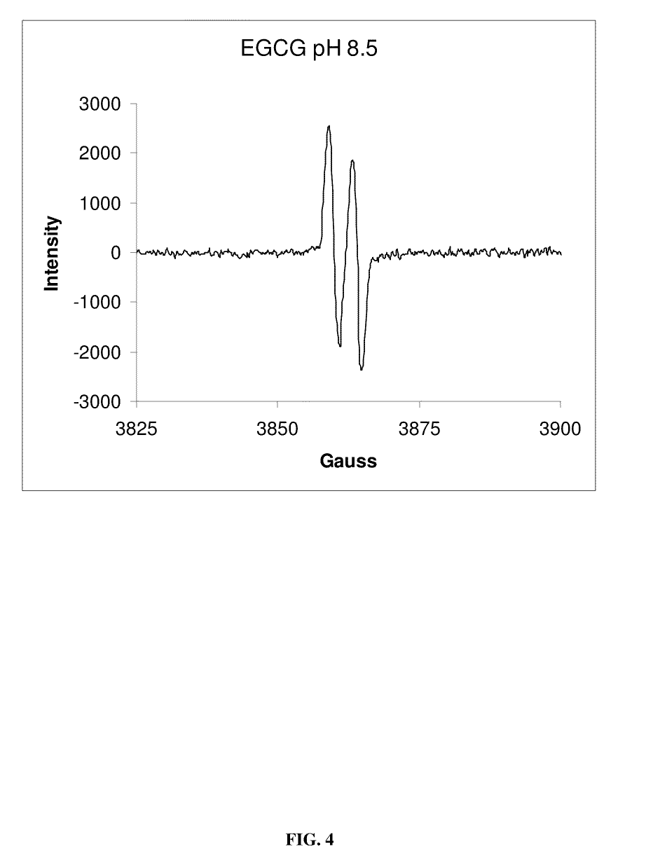

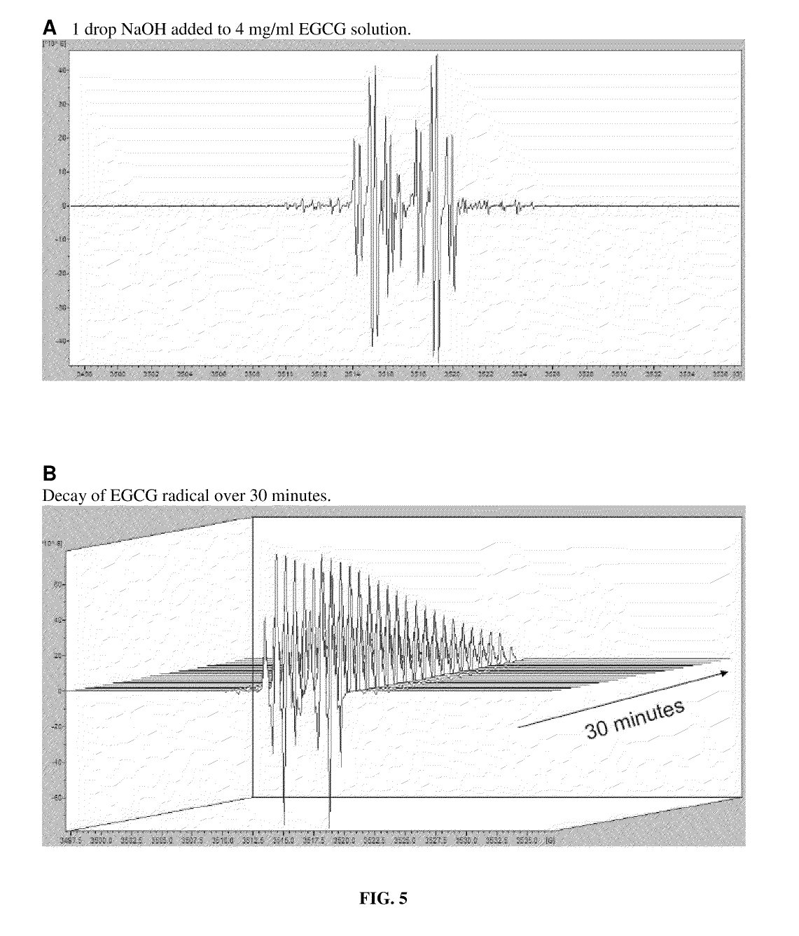

EPR vs. pH study of EGCG: The EPR and UV-visible spectra of EGCG, stabilized with 0.2 M MgCl₂, were measured as a function of pH (i.e., 6.03, 6.54, 7.03, 7.57, 8.09, 8.56, 9.10, 9.56). A corresponding increase in a peak at ˜314 nm and a g=2 EPR spectrum occurred with increasing pH. The addition of MgCl₂ greatly improved the intensity of the EPR spectrum. This is in agreement with the known stabilizing effect on semiquinone free radicals by divalent cations like Mg⁺². FIG. 4 shows the semiquinone radical at pH 8.5. Basification of the EGCG solution resulted in strong semiquinone signals that decayed over 30 minutes, demonstrating radical instability in the absence of electrochemical modulation (FIG. 5). The multiple peaks and valleys in FIGS. 5a and 5b are the fine structure detail of a high concentration radical solution. EPR detects unpaired spinning electrons due to the absorption of microwaves in an applied magnetic field. The peak splitting is due to interaction of the lone electron's spin magnetic field with environmental magnetic fields. In another experiment at pH 6 where no 320 nm peak is ever observed (FIG. 1), a weak EPR signal for the semiquinone was present in the freshly dissolved solution, and the signal increased in intensity as the solution aged to 48 hours. These results demonstrate that the 320 nm peak is not the semiquinone.

### Example 3

Antiviral Activity against Herpes Simplex 1 and 2: Of the many variant forms of herpes viruses, eight are known to affect more than 90% of the world's six billion inhabitants. The three that affect the skin/or mucus membranes are herpes simplex virus 1 (HSV-1, the cold sore virus), herpes simplex virus 2 (HSV-2, the genital herpes virus), and the Varicella zoster virus (VZV, the chicken pox and shingles virus). Herpes simplex viral infections are capable of causing life-threatening and lethal herpes encephalitis in otherwise normal people, lethal and blinding infections in newborns, blindness in adults, and serious to life-threatening infections in immuno-compromised patients. Currently there is no cure for any of the human herpes viral infections. Acyclovir and its derivatives act to interfere with viral reproduction by offering the virus defective nucleoside DNA building blocks that are preferentially incorporated into the replicating viral DNA chain, thus slowing its continued replication. However, these antiviral medications do not kill the herpes virus.

EGCG from green tea has been found to inhibit HSV-1 and HSV-2 (Lyu et al., 2005; Isaacs et al., 2008), influenza virus (Nakayama et al., 1993; Song et al., 2005), adenovirus (Weber et al., 2003), Epstein-Barr virus (Chang et al., 2003), and HIV (Fassina et al., 2002; Yamaguchi et al., 2002; Kawai et al., 2003). In the studies cited above where the mechanism of activity was addressed, EGCG impinged on multiple steps in the life cycle of the virus besides simply blocking absorption. Reduced and oxidized states of EGCG were not addressed in those studies.

Several investigations allude to the relative effects of the antioxidant and pro-oxidant forms of polyphenols and related compounds in antiviral assays. Koyama et al. (2001) found a definite correlation between the redox potentials and inhibitory effects on Epstein-Barr virus activation of azaanthraquinones. Those compounds at the lowest redox potentials at pH 7.2 had the most potent activity, although all the concentrations were high (IC₅₀s>100 μM). Conversely, when the hepatitis C proteinase inhibitor 4-methyl-1-(phenylmethyl)-2,6-pyridinedione underwent an autooxidation process that resulted in dimer formation, the dimer was found to be a relatively more potent inhibitor of the enzyme (Bennett et al., 2005).

Another possibility is that the reduced and oxidized forms of EGCG have different effects in the same cells. In an anticancer study, the auto-oxidation of EGCG led to the inactivation of epidermal growth factor receptor in KYSE 150 cells, but the inhibition of cell growth was observed with reduced EGCG (Hou et al., 2005). Those authors stabilized the EGCG in the reduced state in the growth medium by adding superoxide dismutase, with and without catalase. It is notable that pro-oxidant activity of EGCG has been recorded in studies of a variety of cell culture lines (Alvarez et al., 2002; Tobi et al., 2002; Salter et al., 2004; Elbling et al., 2005). Alvarez et al. (2002) found EGCG acted as a pro-oxidant at low concentrations (1-10 μM) and an antioxidant at higher concentrations, but Tobi et al. (2002) and Salter et al. (2004) found the opposite results: at low concentrations EGCG was an antioxidant, and at high concentrations it was a pro-oxidant.

The inherent ability of EGCG and other polyphenols to act as antioxidants or pro-oxidants, undergo redox reactions with stable phenolic radical intermediates, and chelate metals could be why they have variable bioactivity under in vitro assay conditions (i.e., the in presence of atmospheric O₂). The extent of oxidation (either auto-oxidation or coupled reactions) of EGCG in vivo, where free O₂ is very low, is largely unknown. Sang et al. (2007) found EGCG in the plasma of mice injected with EGCG was conjugated as the 4″-glucoronide, but no oxidized forms of the polyphenol were detected.

EGCG is a good model compound to investigate whether it acts as an antioxidant or pro-oxidant as an antiviral drug under a suite of redox states and pH's. Polyphenols such as EGCG are a promising class of antiviral agents because they appear to inhibit the viruses at multiple steps in their life cycle. A number of synthetic derivatives of natural polyphenols are also under investigation as possible antiviral drugs (Klocking et al., 2002; Savi et al., 2005), and ultimately EGCG may not prove to be the most efficacious antiviral polyphenol.

Tea polyphenols are added to some skin care products and are generally considered to be safe in topical formulas (Thornfeldt, 2005; Hsu, 2005). Treatment of green tea polyphenols to the skin has been shown to modulate the biochemical pathways involved in inflammatory responses, cell proliferation, and responses of chemical tumor promoters as well as ultraviolet light-induced markers of skin inflammation (Katiyer and Elmets, 2001; Kapoor et al., 2004; An et al., 2005). Stability studies of topical formulations have been conducted (Proniuk et al., 2002) and the pharmacokinetics of topical administration of EGCG have been documented (Dvorakova et al., 1999).

Experimental—HSV-1 and 2: Herpes simplex viruses were titrated by inoculation of 10-fold dilutions (HSV-1 was inoculated into Vero cell cultures, and HSV-2 was inoculated into CV1 cultures) in 96-well microtiter tissue culture plates. A virus dilution (0.1 ml) in RPMI 1640 with 1% fetal bovine serum (MM) was inoculated into each well with three wells per dilution. The plates were kept for 2 to 5 days, depending on the virus, and examined daily for cytopathic effect. Virus titers were calculated by the method of Reed and Muench (1938).

About 10⁵ 50% tissue culture infective doses (TCID₅₀s) of virus were mixed with varying concentrations (12.5, 25, 50, 75 and 100 μM) of reduced and oxidized EGCG in MM and incubated at 37° C. for 30 min. The following samples were tested: The reduced form of EGCG (#1); the partially oxidized form of EGCG (#2); the quinone or fully oxidized form of EGCG with the addition of a copper catalyst (#3); and quinone or fully oxidized form of EGCG with the addition of a zinc catalyst (#4). In Samples #3 and #4, the addition of the copper and zinc catalysts, respectively, raised the pH to 9.0-9.5. These compounds were totally oxidized, thereby taking a quinone form. These samples polymerized and turned a dark brown color with a heavy amount of precipitate.

Virus mixed with MM alone was used as a control. After incubation, the infectivity of each mixture was titrated by the serial dilution endpoint method. Dilutions (10-fold) were made in MM. The 10⁻¹ to 10⁻⁵ dilutions were inoculated into monolayers of Vero or CV1 cells, and the virus titers were determined as described above. The difference between the titer (log 10) of the control virus and the titers of EGCG-virus mixtures, i.e., the reduction of virus titer, was used as a measure of antiviral activity.

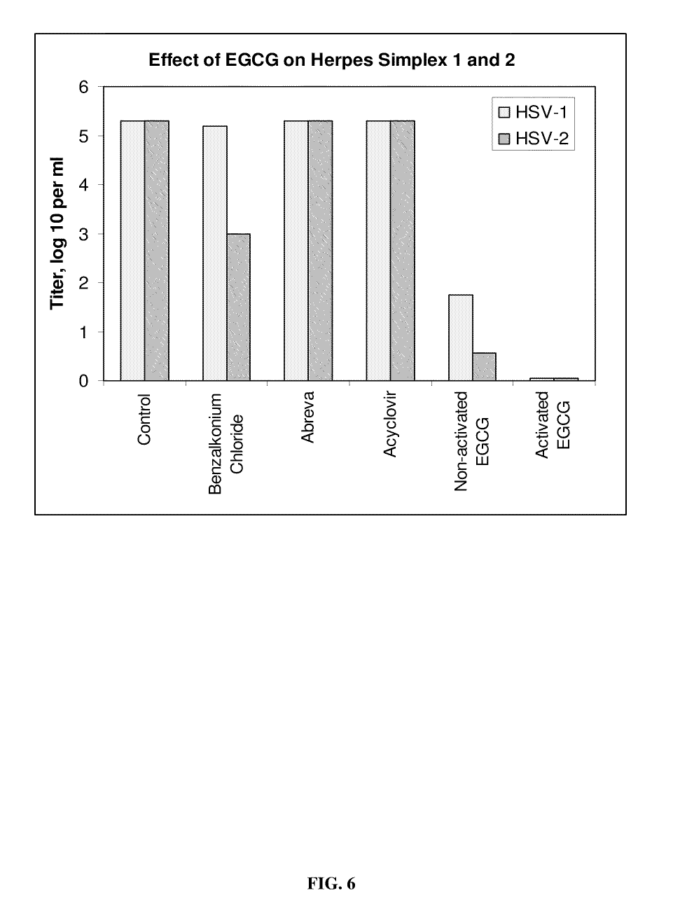

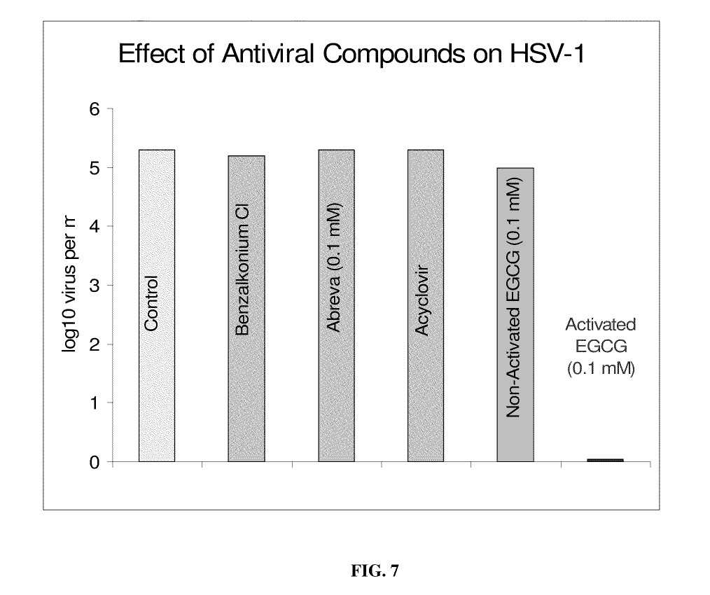

As shown in FIG. 6, the oxidized form of EGCG shows the highest efficacy against HSV-1 and HSV-2 relative to the reduced form compared to commercial viricides. An important follow-up experiment was performed in light of the HSV-1 and -2 data described above. In the antiviral activity assay (FIG. 7) the activated version of EGCG completely inactivated HSV-1 and HSV-2 viruses. The test involves treating suspensions of viruses with a drug, mixing them with the cells they normally infect, incubating the mixture for a preset time that is generally required for viruses to infect the cells and then determining the number of infected cells. The lower number of infected cells represents a better drug. Activated EGCG at a very low concentration (0.1 mM concentration or 40 parts per million) nearly completely inactivated the viruses as only a few infected cells were observed. Other antiviral drugs merely slowed down virus replication but were not able to effectively protect against infection of the test cells. There are plans to test EGCG against four members of the herpes family: HSV-1, HSV-2, human cytomegalovirus, and varicella zoster virus (chicken pox and shingles). In on-going experiments, EGCG inactivated the herpes family of viruses at very low concentration compare to the concentration that impacted the test cells. The low concentrations that inactivate the viruses, coupled with the high safety level against the host cells make EGCG a very promising drug candidate. Generally most of the test compounds that work against the viruses are also highly toxic to host cells, as shown in Table 1 below.

TABLE 1
Relative Toxicity of EGCG with Respect to Virus Type

| | Antiviral Drug Screens of | | | |
|---|---|---|---|---|
| | EGCG Antiviral activity | | | |
| | μg/ml | μg/ml | μg/ml | |
| Viral Target | EC-50 | EC-90 | CC-50 | SI |
| HSV-1 (herpes | 24.1 | 121 | 857 | 35.5 |
| simplex 1) | | | | |
| HSV-2 (herpes | 7.6 | 98.6 | 857 | 113 |
| simplex 2) | | | | |
| HCMV (human | 6.9 | 40 | >40 | >5.8 |
| cytomegalovirus) | | | | |
| VZV (varicella | 4.1 | >40 | >40 | >9.7 |
| zoster virus) | | | | |
| TOXICITY | Neutral | Test Sample | GCV | ACV |
| | Red | CC50 > 200 | CC50 > 100 | CC50 > 100 |
| | Uptake | | | |

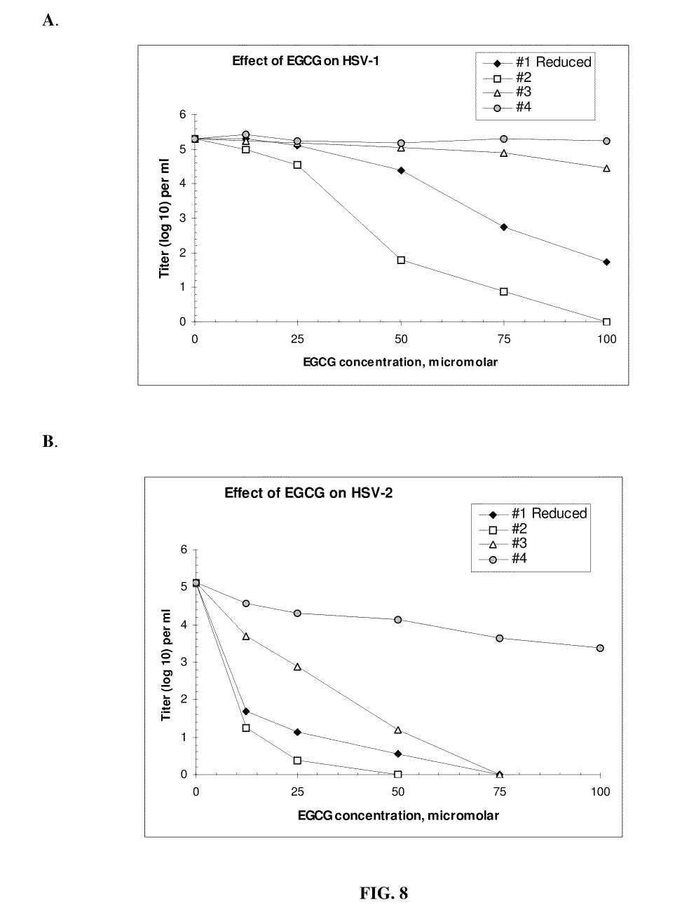

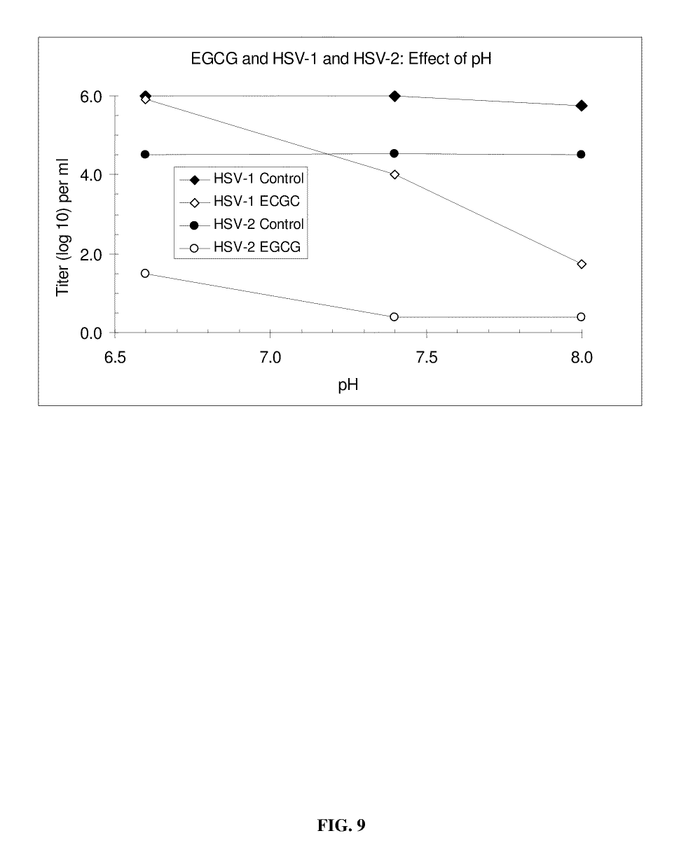

FIGS. 8A and 8B demonstrate that the pro-oxidant forms of EGCG are more effective against HSV-1 and HSV-2, respectively, and FIG. 9 shows greater antiviral activity at the more alkaline pH's when semiquinone, quinone, and oligomeric forms of EGCG are present. The concentrations of EGCG used in these assays (0-100 μM) were not cytotoxic to the host cells (Table 2).

TABLE 2
Reduced and Oxidized EGCG are not Toxic to Vero Cells

| | | Time Following EGCG Addition to Vero Cells (Hours) | | |
|---|---|---|---|---|
| | | 0 | 24 | 48 |
| Control | | 0.630 ± 0.025^{1,2} | 0.702 ± 0.044 | 0.831 ± 0.040 |
| Reduced EGCG | (50 μM) | — | 0.753 ± 0.027 | 0.750 ± 0.033 |
| | (75 μM) | — | 0.685 ± 0.052 | 0.706 ± 0.026 |
| | (100 μM) | — | 0.700 ± 0.043 | 0.705 ± 0.019 |
| Oxidized EGCG | (50 μM) | — | 0.699 ± 0.013 | 0.731 ± 0.010 |
| | (75 μM) | — | 0.708 ± 0.022 | 0.729 ± 0.007 |
| | (100 μM) | — | 0.674 ± 0.013 | 0.696 ± 0.017 |

¹These assays were performed using the Cell Titer 96 Aqueous Cell Proliferation Assay from Promega. It is accomplished by dehydrogenase enzymes in metabolically active cells.
²Each number is the mean ± SD for assays done in triplicate.

### Example 5

Antiviral activity against HIV-1: EGCG is a known inhibitor of human immunodeficiency virus HIV-1 (Fassina et al., 2002; Yamaguchi et al., 2002; Kawai et al., 2003). The AIDS epidemic caused by the human immunodeficiency virus HIV-1 has no cure. Antiviral drugs and drug combinations slow viral replication, but don't eliminate the virus. In the underdeveloped world, the cost of the current drugs is prohibitive, and the death rates among certain populations have changed the fabric of societies. For example, it is estimated worldwide that more than 15 million children under the age of 18 have been orphaned as a result of AIDS. More than 12 million of these children live in Sub-Saharan Africa, where it is currently estimated that 9% of all children have lost at least one parent to AIDS (http://www.avert.org/aidsorphans.htm). As HIV infections become increasingly common among the heterosexual adult population of the region, millions of children will lose parents to AIDS. By 2010, it is predicted that there will be around 15.7 million AIDS orphans in Sub-Saharan Africa alone. The technology embodied in this patent can be developed to produce an effective topical viricide that kills HIV-1

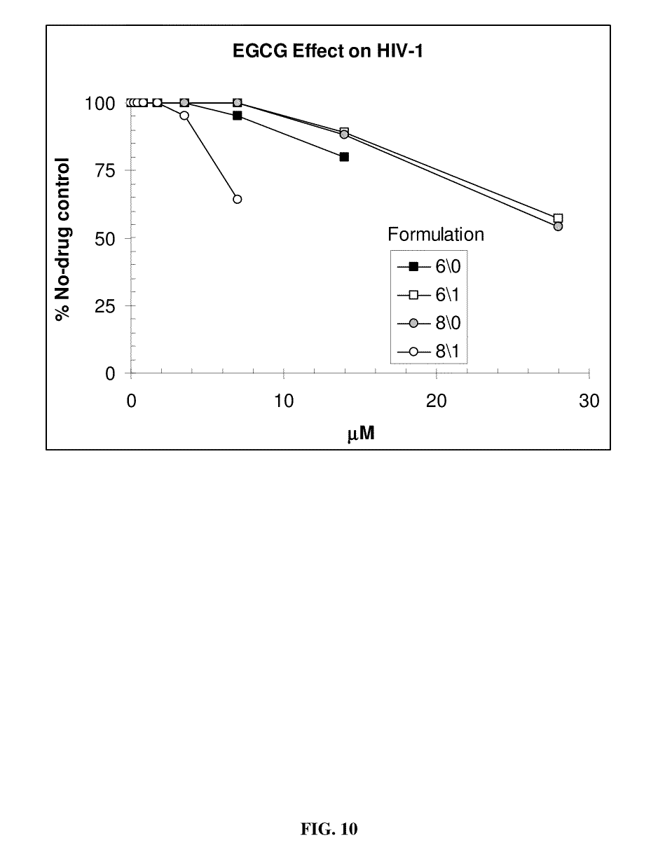

FIG. 10 demonstrates the activity profiles of reduced and oxidized solutions of EGCG against HIV-1 suspensions before infecting cells. The most oxidized formulation was the most effective.

### Example 6

Inhibition of apoptosis is implicated in virtually every known human malignancy (Reed, 1995; Johnstone et al., 2002). Proteins in the Bcl-2 (B-cell lymphocyte/leukemia-2) family are critical components of the intrinsic apoptotic pathway, and anti-apoptotic Bcl-2 proteins such as Bcl-xL are over-expressed in most human cancer types. Inhibition of apoptosis may involve more than one pathway. In MIA PaCa-2 pancreatic carcinoma cells, EGCG invokes Bax oligomerization and depolarization of mitochondrial membranes to facilitate cytochrome c release into cytosol and caspase-dependent apoptosis (Qanungo et al., 2005). Those authors concluded that EGCG induces stress signals by damaging mitochondria and reactive oxygen species-mediated JNK activation in pancreatic cancer cells. Nakagawa et al. (2004) concluded that the generation of hydrogen peroxide by EGCG primarily contributes to the induction of Fe(II)-dependent apoptosis in Jurkat cells, indicating a beneficial pro-oxidant mechanism.

From a combination of NMR binding assays, fluorescence polarization assays, and computational-docking studies, it is known that the green tea compounds EGCG, gallocatechin-3 gallate (GCG), epicatechin-3 gallate (ECG) and catechin-3 gallate (CG) are very potent inhibitors (Kᵢs in the nanomolar range) of the anti-apoptotic protein Bcl-xL (Kitada et al., 2003). The inherent ability of EGCG to act as antioxidant or pro-oxidant, undergo redox reactions with stable phenolic radical intermediates, and chelate metals could be why it is bioactive once it is bound to a target.

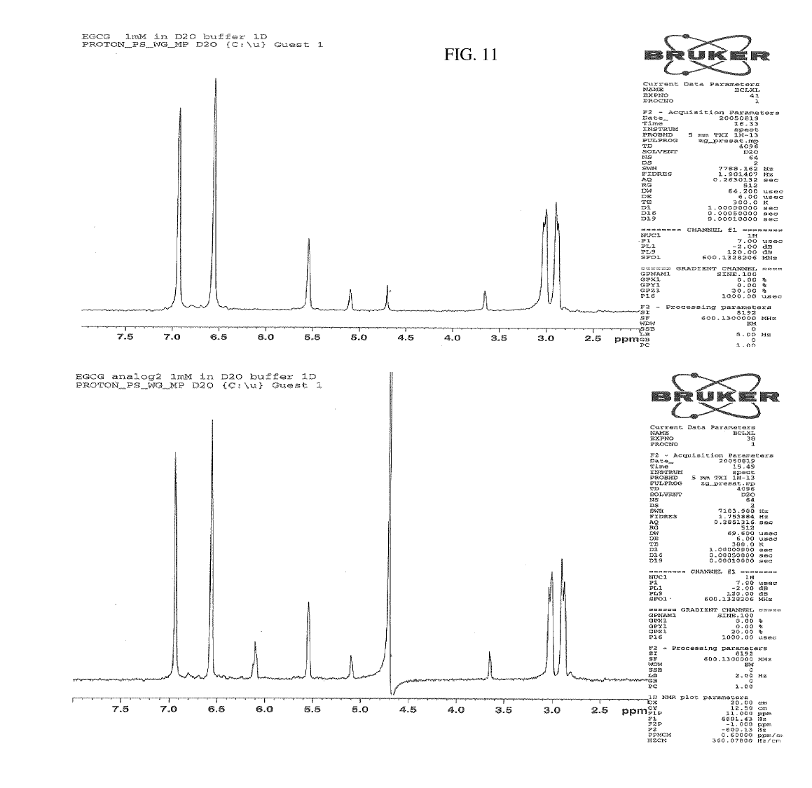

In this Example using the Bcl-xL binding assays to support our claims, EGCG solutions were the same as those used in some of the antiviral assays (FIGS. 8A and 8B), including reduced, partially oxidized, oxidized containing Cu²⁺, and oxidized containing Zn²⁺ under assay conditions outlined by Pellechia et al. (2002) and Pellechia and Reed (2004). 1D-¹H NMR spectra of all the compounds were taken at 1 mM concentration, 1% D₂O, 10% H₂O, 50 mM phosphate buffer at pH near 7.2. The partially oxidized EGCG solution without metal ions showed a two-fold better binding response in the fluorescence polarization displacement assay with a FITC-Bad peptide, and the NMR spectrum of Bcl-xL had an extra peak at 6.1 ppm (FIG. 11). The two other highly oxidized preparations of EGCG showed either no difference or less activity than reduced EGCG. Gossypol, another polyphenol with known anticancer activity, was also tested under similar conditions. The solution of partially oxidized gossypol had similar activity as reduced EGCG (it is normally several times less active than EGCG in the same assay). It produced a similar NMR spectrum as reduced gossypol with a single aromatic peak, but the peak typical of the free aldehyde had a marked decreased intensity. These data demonstrate that oxidized states of EGCG and gossypol may be more active agents for binding to the anti-apoptosis protein, and that redox states of activity can be monitored by spectrometric methods. The Bcl-xL assay is an appropriate model system to show feasibility and proof of concept that redox control of anticancer drug agents can improve their activity.

### Example 7

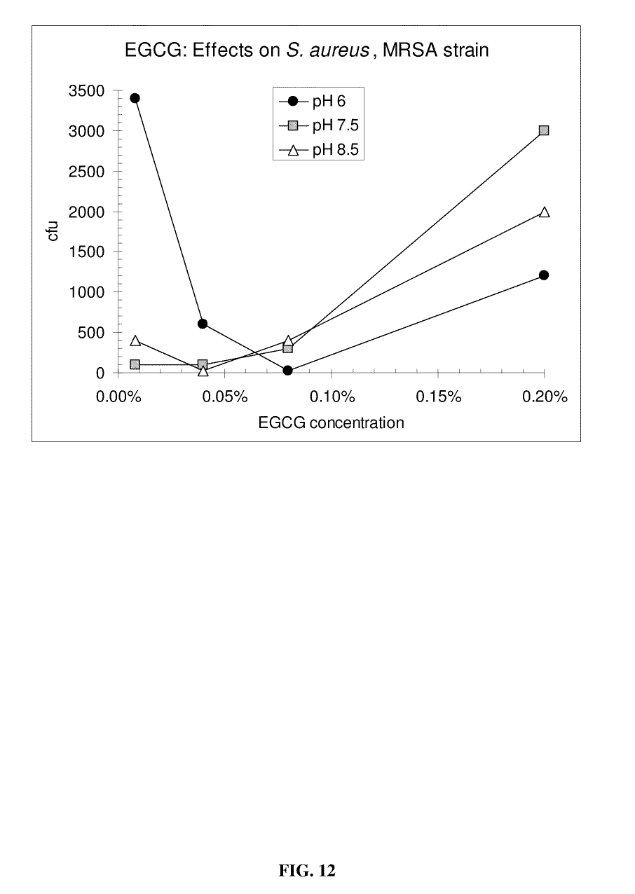

Antibacterial activity: EGCG was tested as an antibacterial agent against a multidrug-resistant strain (33591) of Staphylococcus aureus. After pre-incubating the cells with various concentrations of EGCG at pH 6, 7.5, and 8.5, cells were plated and colony-forming units (cfu) were counted as a measure of efficacy (FIG. 12). At the lowest concentration tested (0.08% EGCG), only the solutions containing pro-oxidant semiquinone and quinone forms (pH 7.5 and 8.5) were effective at killing nearly all the bacteria. At concentrations of 0.4-0.8% EGCG, the solutions were strongly bactericidal at all three pH's. At higher concentrations, EGCG was less effective. With the burgeoning medical problem of drug-resistant pathogens surviving all available drug therapies, the discovery of highly effective polyphenolic drug candidates whose efficacy can be modulated by redox and pH falls under the claims of this patent.

### Example 8

Oxidation States Modulated and Measured Electrochemically: The multiple oxidation states of the molecule of interest can be determined electrochemically by cyclic voltammetry. The first phase involves applying an excessive positive potential (oxidizing potential) and oxidizing all the species present to one form. Then the applied voltage is stepped down in increments while monitoring current. When the mid-point redox potential of a reaction is approached, molecules begin to be reduced, causing current through the circuit. After returning all molecules to the reduced form, the voltage is increased and the redox potentials of the reaction(s) are recorded. This technique has been employed by others to determine redox states of EGCG (Kilmartin and Hsu, 2003).

Additionally, the open circuit potential of the solution versus a standard reference electrode can be measured. This method utilizes two electrodes, working and reference, to determine the electrochemical potential generated by the solution versus a reference electrode, such as Ag/AgCl. This method will give an indication of the overall degree of oxidation of the formulation, and when combined with cyclic voltammetry, can be used to determine the species present.

By knowing the electrochemical potentials that create the semiquinone and quinone species, an external electrical potential can be applied to drive the redox state of the formulation to a predetermined oxidation state. Continued application of the electrical potential will maintain the formulation in the desired oxidation state. This electrochemical potential can be supplied from a battery, small circuit, and electrodes designed into a container format (battery-in-a-bottle). The reduction potential of EGCG at pH 7 is 430 mV versus a normal hydrogen electrode.

An electrochemical cell was fabricated using a fine porosity glass frit in one-inch-diameter glass tubing. The electrochemical cell houses the counter electrode and keeps the Ag/AgCl reference electrode and platinum working electrode separate from the counter electrode. This addition to the system allowed the preparation of bulk solution. The working electrode and reference electrode compartment was continuously stirred using a small stir bar and a magnetic stirrer to minimize gradient effects at the working electrode.

The EGCG solutions were oxidized using a positive potential to a visual end point. UV-visible spectral data were collected during these experiments, including the absorbance at 270 nm and 320 nm, corresponding to the phenol and quinone forms of EGCG, respectively. In addition, the open cell potential was recorded as experiments progressed. The active redox potentials and experimental endpoints were determined based on current and visual color changes to oligomers.

Due to solution evaporation over time, the ratio of absorbance at 320 nm divided by the absorbance at 270 nm was determined in order to account for overall absorption increases. This value was then tracked throughout the experiments and proved to be a more stable indicator of reaction progress than absorbance alone. In addition, the visual change in solution color from clear to yellow and pink was also monitored as an indicator of degree of oxidation.

Stirred EGCG solutions in 0.1 M phosphate buffer with 0.1 M KCl at pH 6.0 were monitored. The open circuit potential was initially 220 mV. A potential of 800 mV was applied, resulting in approximately 1 microamp of current in the cell. A duplicate solution was left uncovered on the table next to the electrochemical cell as the control. After seven hours, no change in either solution at 270 and 320 nm was found. At this point the experimental potential was increased to 1000 mV for a further 16 hours (Table 3A).

These data demonstrate the increase in 320 nm quinone absorbance and associated decrease in the 270 nm phenol absorbance associated with oxidation of the EGCG over 16 hours at 1000 mV. The control sample did not oxidize, which is consistent with the known stability of EGCG under acidic conditions. After the combined time of 23 hours (7 h plus 16 h), the potential of the cell was increased to 1500 mV (Table 3B).

At 68 hours, the control sample was still clear, but the experimental solution had a faint yellowish color (dimerization). The pH of the solutions remained 6.0 throughout the experiment. All data support the forced oxidation of EGCG at pH 6.0. Solutions were allowed to sit for two more days to observe the stability of the electrochemically-induced changes (Table 3C).

TABLE 3
Electrochemical modification of EGCG

| | | | | |
|---|---|---|---|---|
| Sample ID | Time | 270 nm | 320 nm | 320 nm/270 nm |

A

| | | | | |
|---|---|---|---|---|
| Experiment | 23 hours | 0.418 | 0.107 | 0.256 |
| Control | 23 hours | 0.424 | 0.072 | 0.170 |

B

| | | | | |
|---|---|---|---|---|
| Control | 43 hours | 0.448 | 0.078 | 0.174 |
| Experiment | 43 hours | 0.432 | 0.171 | 0.396 |
| Control | 55 hours | 0.471 | 0.084 | 0.178 |
| Experiment | 55 hours | 0.422 | 0.197 | 0.467 |
| Control | 68 hours | 0.438 | 0.083 | 0.189 |
| Experiment | 68 hours | 0.429 | 0.224 | 0.522 |

C

| | | | | |
|---|---|---|---|---|
| Experiment | 73 hours | 0.430 | 0.229 | 0.533 |
| Experiment | 79 hours | 0.426 | 0.229 | 0.538 |
| Experiment | 120 hours | 0.436 | 0.238 | 0.546 |

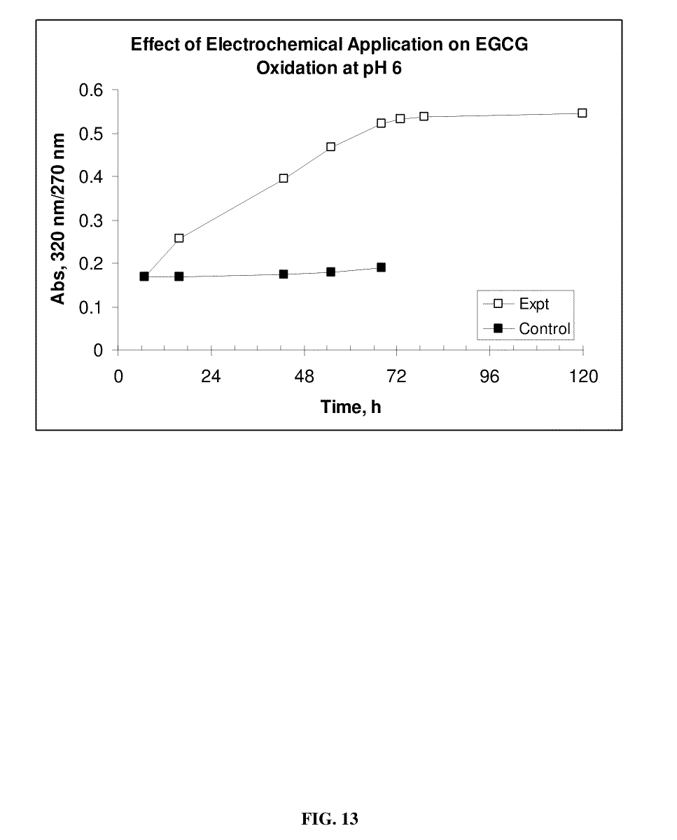

These data indicate that the solutions are stable for over two days without a significant increase or decrease in 320 nm absorbance over time. A summary of these data is presented in FIG. 13.

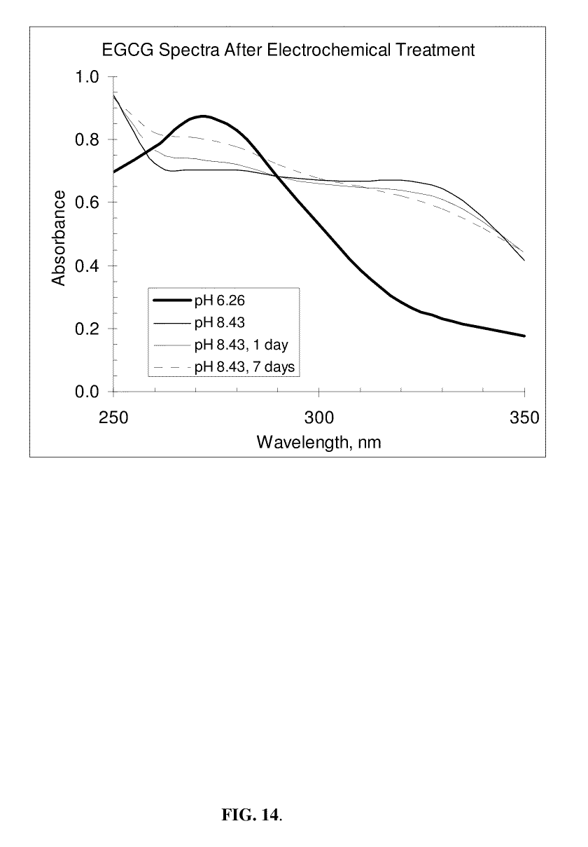

Solutions were pH-modified following electrochemical oxidation to observe stability. The spectra of samples prepared from an EGCG solution at pH 6.26 under 1500 mV potential for 11.4 hours are shown in FIG. 14. The pH 6.26 solutions could not be compared to control samples because control samples at pH 6.26 did not oxidize. The figure shows the samples were stable after one day, and in the case of pH 8.43, it was relatively stable after 7 days. These samples were open to air, and no other precautions were taken to prevent oxidation by atmospheric oxygen.

The following references are specifically applicable to the Examples and to the remainder of the specification. They are incorporated herein by referenced and are identified, where appropriate, by their authors and publication dates as indicated.

### REFERENCES

Alvarez E, Leiro J, Orallo F. 2002. Effect of (−)-epigallocatechin-3-gallate on respiratory burst of rat macrophages. Int Immunopharmacol. 2 (6): 849-855.
An B J, Kwak J H, Son J H, Park J M, Lee J Y, Kim Y S, Jo C, Byun M W. 2005. Physiological activity of irradiated green tea polyphenol on the human skin. Am J Chinese Med. 33 (4): 535-546.
Asanuma, M, I Miyazaki, et al. 2003. Dopamine- or L-DOPA-induced neurotoxicity: the role of dopamine quinone formation and tyrosinase in a model of Parkinson's disease. Neurotox. Res. 5: 165-176.
Bors, W, C Michel, and K Stettmaier. 2000. Electron paramagnetic resonance studies of radical species of proanthocyanidins and gallate esters. Arch. Biochem. Biophys. 374: 347-355.
Chang L K, Wei T T, Chiu Y F, Tung C P, Chuang J Y, Hung S K, Li C, Liu S T. 2003. Inhibition of Epstein-Barr virus lytic cycle by (−)-epigallocatechin gallate. Biochem Biophys Res Commun. 301(4):1062-8.
Dvorakova K, Dorr R T, Valcic S, Timmermann B, Alberts D S. 1999. Pharmacokinetics of the green tea derivative, EGCG, by the topical route of administration in mouse and human skin. Cancer Chemother Pharmacol. 43 (4): 331-335.
Elbling L, Weiss R M, Teufelhofer O, Uhl M, Knasmueller S, Schulte-Hermann R, Berger W, Mickshe M. 2005. Green tea extract and (−)-epigallocatechin-3-gallate, the major tea catechin, exert oxidant but lack antioxidant activities. FASEB J 19 (2): U17-U42.
Fassina G, Buffa A, Benelli R, Varnier O E, Noonan D M, Albini A. 2002. Polyphenolic antioxidant (−)-epigallocatechin-3-gallate from green tea as a candidate anti-HIV agent. AIDS 16:939-41.
Hamza, A, and C-G Zhan. 2006. How can (_)-epigallocatechin gallate from green tea prevent HIV-1 infection? Mechanistic insights from computational modeling and the implication for rational design of anti-HIV-1 entry inhibitors. J. Phys. Chem. B 110:2910-2917.
Hou Z, Sang S, You H, Lee M J, Hong J, Chin K V, Yang C S. 2005. Mechanism of action of (−)-epigallocatechin-3-gallate: auto-oxidation-dependent inactivation of epidermal growth factor receptor and direct effects on growth inhibition in human esophageal cancer KYSE 150 cells. Cancer Res. 65:8049-56.
Hsu S. 2005. Green tea and the skin. J Am Acad Dermatol. 52 (6): 1049-1059.
Isaacs, C E, G Y Wen, W Xu, J H Jia, L Rohan, C Corbo, V Di Maggio, E C Jenkins, Jr., and S Hillier. 2008. Epigallocatechin gallate inactivates clinical isolates of herpes simplex virus. Antimicrob. Agents Chemother. 52: 962-970.
Johnstone, R W, A A Ruefli, and S W Lowe. 2002. Apoptosis: a link between cancer genetics and chemotherapy. Cell 108: 153-164.
Kapoor M, Howard R, Hall I, Appleton I. 2004. Effects of epicatechin gallate on wound healing and scar formation in a full thickness incisional wound healing model in rats. Am J. Pathol. 165 (1): 299-307.
Katiyar S K, Elmets C A. 2001. Green tea polyphenolic antioxidants and skin photoprotection (Review). Int J. Oncol. 18 (6): 1307-1313.
Kawai K, Tsuno N H, Kitayama J, Okaji Y, Yazawa K, Asakage M, Hori N, Watanabe T, Takahashi K, Nagawa H. 2003. Epigallocatechin gallate, the main component of tea polyphenol, binds to CD4 and interferes with gp120 binding. J Allergy Clin Immunol. 112:951-7.
Kilmartin, P A, and C F Hsu, 2003. Characterisation of polyphenols in green, oolong, and black teas, and in coffee, using cyclic voltammetry. Food Chem. 82: 501-512.
Kitada, S, M Leone, S Sareth, D Zhai, J C Reed, and M Pellecchia. 2003. Discovery, characterization and structure activity relationship studies of pro-apoptotic polyphenols targeting Bcl-xL. J. Med. Chem. 46: 4259.
Klocking R, Helbig B, Schotz G, Schacke M, Wutzler P. 2002. Anti-HSV-1 activity of synthetic humic acid-like polymers derived from p-diphenolic starting compounds. Antivir Chem Chemother. 13(4): 241-9.
Koyama J, Morita I, Tagahara K, Osakai T, Hotta H, Yang M X, Mukainaka T, Nishino H, Tokuda H. 2001. Correlation with redox potentials and inhibitory effects on Epstein-Barr virus activation of azaanthraquinones. Chem Pharm Bull (Tokyo). 49:1214-6.
Lyu S Y, Rhim J Y, Park W B. 2005. Antiherpetic activities of flavonoids against herpes simplex virus type 1 (HSV-1) and type 2 (HSV-2) in vitro. Arch Pharm Res. 28:1293-1301.
Mochizuki, M, S-i Yamazaki, K Kano, and T Ikeda. 2002. Kinetic analysis and mechanistic aspects of autoxidation of catechins. Biochim. Biophys. Acta/Gen. Subj. 1569: 35-44.
Nakagawa, H, K Hasumi, J-T Woo, K. Nagai, and M. Wachi. 2004. Generation of hydrogen peroxide primarily contributes to the induction of Fe(II)-dependent apoptosis in Jurkat cells by (−)-epigallocatechin gallate. Carcinogenesis 25: 1567-1574.
Nakayama M, Suzuki K, Toda M, Okubo S, Hara Y, Shimamura T. 1993. Inhibition of the infectivity of influenza virus by tea polyphenols. Antiviral Res. 21:289-99. Pellecchia, M., D. Sem, and K. Wüthrich. 2002. NMR in drug discovery. Nat. Rev. Drug Disc. 1: 211-218.
Pellecchia, M, and J C Reed. 2004. Inhibition of anti-apoptotic Bcl-2 family proteins by natural polyphenols. New avenues for cancer chemoprevention and chemotherapy. Curr. Pharmaceut. Design 10: 1387-1398.
Potta, S P., M X Doss, J Hescheler, and A Sachinidis. 2005 Epigallocatechin-3-gallate (EGCG): A structural target for the development of potential therapeutic drugs against anti-proliferative diseases. Drug Design Rev.-Online 2: 85-91.
Proniuk S, Liederer B M, Blanchard J. 2002. Preformulation study of epigallocatechin gallate, a promising antioxidant for topical skin cancer prevention. J Pharmaceut Sci. 91 (1): 111-116.
Qanungo, S, M Das, S Haldar, and A Basu. 2005. Epigallocatechin-3-gallate induces mitochondrial membrane depolarization and caspase-dependent apoptosis in pancreatic cancer cells. Carcinogenesis 26: 958-967.
Reed, J C. 1995. Regulation of apoptosis by bcl-2 family proteins and its role in cancer and chemoresistance. Current Opinion Oncology 6: 541-546.
Reed, L J and Muench, M. 1938. A simple method of estimating 50 percent end points. Am. J. Hyg. 27; 493-497.
Salter L, Clifford T, Morley N, Gould D, Campbell S, Curnow A. 2004. The use of comet assay data with a simple reaction mechanism to evaluate the relative effectiveness of free radical scavenging by quercetin, epigallocatechin gallate and N-acetylcysteine in UV-irradiated MRC5 lung fibroblasts. J Photochem Photobiol B-Biol. 75 (1-2): 57-61.
Sang, S, I Yang, B Buckley, C-T Ho, and C S Yang. 2007. Autoxidative quinone formation in vitro and metabolite formation in vivo from tea polyphenol (−)-epigallocatechin-3-gallate: Studied by real-time mass spectrometry combined with tandem mass ion mapping. Free Radical Biology & Medicine 43:362-371.
Savi L A, Leal P C, Vieira T O, Rosso R, Nunes R J, Yunes R A, Creczynski-Pasa T B, Barardi C R, Simoes C M. 2005. Evaluation of anti-herpetic and antioxidant activities, and cytotoxic and genotoxic effects of synthetic alkyl-esters of gallic acid. Arzneimittelforschung. 55(1):66-75.
Song, J M, Lee K H, Seong B L. 2005. Antiviral effect of catechins in green tea on influenza virus. Antiviral Res. 68:66-74.
Su, Y L, L K Leunga, Y Huang, and Z-Y Chen. 2003. Stability of tea theaflavins and catechins. Food Chem. 83: 189-195.
Thornfeldt, C. 2005. Cosmeceuticals containing herbs: Fact, fiction, and future. Dermotol Surg. 31 (7): 873-880 Part 2.
Tobi S E, Gilbert M, Paul N, McMillan T J. 2002. The green tea polyphenol, epigallocatechin-3-gallate, protects against the oxidative cellular and genotoxic damage of UVA radiation. Int J Cancer 102 (5): 439-444.
Van Maanen, J J, M V Lafleur, D R Mans, E van den Akker, C de Ruiter, P R Kootstra, D Pappie, J de Vries, J Retel, and H M Pinedo. 1988. Effects of the ortho-quinone and catechol of the antitumor drug VP-16-213 on the biological activity of single-stranded and double-stranded phi X174 DNA. Biochem Pharmacol. 37: 3579-3589.
Weber J M, Ruzindana-Umunyana A, Imbeault L, Sircar S. 2003. Inhibition of adenovirus infection and adenain by green tea catechins. Antiviral Res. 58: 167-73.
Yamaguchi K, Honda M, Ikigai H, Hara Y, Shimamura T. 2002. Inhibitory effects of (−)-epigallocatechin gallate on the life cycle of human immunodeficiency virus type 1 (HIV-1). Antiviral Res. 53:19-34.
Zhou, Q, H Chiang, C Portocarrero, Y Zhu, S Hill, K Heppert, H Jayaratna, M Davies, E Janle, and P Kissinger. 2003. Investigating the stability of EGCg in aqueous media. Curr. Separations 20:83-86.

We claim:

1. A composition with antiviral and antibacterial activity comprised of at least one oxidized compound and an oxidizing agent.

2. The composition of claim 1, wherein the oxidized compound is present in a quantity between 0.01% and 40% by weight and the oxidizing agent is present in a quantity between 0.001% and 10% by weight.

3. The composition of claim 1, wherein the oxidized compound is EGCG.

4. The composition of claim 1, wherein the oxidized compound is capable of having an enhanced property, further wherein the enhanced property is a member selected from the group consisting of antiviral, antibacterial and disinfectant.

5. The composition of claim 2, wherein the oxidized compound capable of having antiviral properties is effective in killing a virus selected from the group consisting of herpes virus and human immunodeficiency virus.

6. The composition of claim 1, wherein the oxidizing agent is a transition metal ion.

7. The composition of claim 1, wherein the oxidized compound is present in a quantity between 0.005% and 12% by weight.

8. The composition of claim 1, wherein the oxidized compound is a phenol.

9. The composition of claim 1, wherein the oxidized compound is a polyphenol.

10. The composition of claim 1, wherein the oxidized compound is an antioxidant.

11. The composition of claim 1, wherein the oxidized compound is redox-active.

12. The composition of claim 1, wherein the oxidized compound is in a form selected from the group consisting of semiquinone and quinone.

13. The composition of claim 11, wherein the form of the oxidized compound is created by increasing the pH greater than 7.0.

14. The composition of claim 11, wherein the form of the oxidized compound is created electrochemically.

15. The composition of claim 1, wherein the oxidizing agent is present in a quantity between 0.05% and 2.0% by weight.

16. A method of treating cells infected with a virus comprising administering an effective amount of an oxidized form of a polyphenolic to the infected cells.

17. The method of claim 14, wherein the polyphenolic is in a semiquinone form.

18. The method of claim 14, wherein the polyphenolic is in a quinone form.
